(19) United States
(12) Patent Application Publication
(10) Pub. No.: US 2025/0346546 A1
(43) Pub. Date: Nov. 13, 2025

(54) METHOD AND APPARATUS FOR PURIFYING GASEOUS PRODUCTS FROM A CO2 ELECTROLYSIS PROCESS

(71) Applicant: Siemens Energy Global GmbH & Co. KG, Munich, Bayern (DE)

(72) Inventors: Ralph Joh, Seligenstadt (DE); Wiebke Sarfert-Gast, Herzogenaurach (DE); Günter Schmid, Hemhofen (DE); Rüdiger Schneider, Eppstein (DE)

(73) Assignee: Siemens Energy Global GmbH & Co. KG, Munich, Bayern (DE)

(21) Appl. No.: 18/862,644

(22) PCT Filed: May 4, 2023
(86) PCT No.: PCT/EP2023/061781

§ 371 Date: Nov. 4, 2024

(30) Foreign Application Priority Data
May 11, 2022 (DE) 10 2022 204 626.9

(51) Int. Cl. C07C 7/04; B01D 7/00; B01D 53/22; C25B 1/23; C25B 3/03; C25B 3/26; C25B 15/08

(52) U.S. Cl. CPC C07C 7/04; B01D 7/00; B01D 53/229; C25B 15/083; B01D 2256/24; B01D 2257/108; B01D 2257/502; B01D 2257/504; B01D 2257/80; C25B 1/23; C25B 3/03; C25B 3/26

(57) ABSTRACT

A method for treating products from a CO₂ electrolysis process where carbon dioxide (CO₂) and water (H₂O) are electrochemically reacted in a cathode chamber of an electrolysis cell. Gaseous cathode products are formed which comprise ethylene (C₂H₄), hydrogen (H₂) and carbon monoxide (CO). The products are treated in a multi-stage separating process. First, the cathodic product gas stream is fed to a desublimation process, where CO₂ and water (H₂O) are frozen out of, and separated from, the product gas stream. Next, the product gas stream is compressed to a pressure, and then the compressed product gas stream is fed to a gas permeation process, wherein hydrogen (H₂) is separated off by passing the hydrogen through a hydrogen-permeable membrane. Next, the retentate which remains and which comprises ethylene (C₂H₄) and carbon monoxide (CO) is subjected to distillative separation, such that ethylene (C₂H₄) and carbon monoxide (CO) are separated.

### CROSS REFERENCE TO RELATED APPLICATIONS

This application is the US National Stage of International Application No. PCT/EP2023/061781 filed 4 May 2023, and claims the benefit thereof, which is incorporated by reference herein in its entirety. The International Application claims the benefit of German Application No. DE 10 2022 204 626.9 filed 11 May 2022.

### FIELD OF INVENTION

The invention relates to a method for purifying gaseous products from a CO₂ electrolysis. The invention furthermore relates to an apparatus for purifying gaseous products from the CO₂ electrolysis.

### BACKGROUND OF INVENTION

Currently, about 80% of the energy demand worldwide is met by the combustion of fossil fuels. By these combustion processes and further industrial production processes, about 38.017 million tonnes of carbon dioxide (CO₂) were released into the atmosphere worldwide in the year 2019 (European Commission, Joint Research Centre, Crippa, M., Guizzardi, D., Muntean, M. et al., Fossil CO₂ and GHG emissions of all world countries: 2020 report, Publications Office, 2020, https://data.europa.eu/doi/10.2760/56420).

Besides fossil energy generation, the production and conversion of industrial raw materials is also making a significant contribution to the continuous increase of the CO₂ concentration in the atmosphere, with a fraction of 9.2% of the total emission (Data for Climate Action-Historical GHG Emissions-Global Historical Emissions. In: Climate Watch. World Resources Institute (WRI)). The debate over the negative effects of the greenhouse gas CO₂ on the climate has led to the recycling of CO₂ being envisioned. In thermodynamic terms, CO₂ has a very low value and can therefore be reduced again to form usable products only with difficulty.

In nature, CO₂ is converted into carbohydrates by photosynthesis. This process, which in terms of time and at the molecular level in terms of space is comprised of many substeps, can be commercially replicated only with great difficulty. The electrochemical reduction of CO₂ represents the currently more efficient route in comparison with pure photocatalysis. As in the case of photosynthesis, in this process CO₂ is converted into a higher-energy product (such as CO, CH₄, C₂H₄, etc.) by supplying electrical energy, which is obtained from renewable energy sources such as wind or solar. The amount of energy required during this reduction corresponds in the ideal case to the combustion energy of the fuel and should only come from renewable sources, or use electricity that specifically cannot be taken from the grid. However, surplus production of renewable energy is available not continuously but temporarily only at times with intense sunshine and strong wind. This will be reinforced even more in the near future with the further development of renewable energy.

As in the case of many processes which are set in motion and enhanced catalytically, the chemical reactions involved do not take place selectively but instead there is a product spectrum of different components that are formed. Furthermore, the reactions do not take place quantitatively but result in product flows which consist of a mixture of unreacted starting materials and final products or byproducts. These mixed and nonspecific product flows are industrially and commercially unusable in this form, and need to be separated into their constituents. The isolated starting products may be recirculated into the electrolysis process.

Depending on the task, chemical materials are in general respectively required with very high purity (>99.9%). Here, it is important to find and combine suitable separation steps/methods so that, on the one hand, it is possible to attain a high purity and a high yield, and on the other hand the energy expenditure required therefor is as low as possible, particularly in order to achieve separation and specific selection of the desired products on the industrial scale.

Previous Solution Approaches from the Prior Art

Extensive systematic studies into the electrochemical reduction of CO₂ did not take place until the 1970s. Despite many endeavors, no electrochemical system by which, with a sufficiently high current density and acceptable yield, it is possible to reduce CO₂ in a longterm-stable and energetically favorable fashion into competitive energy carriers has yet successfully been developed. Because of the increasing resource constraint of fossil fuels and the unpredictable availability of renewable energy sources, research into CO₂ reduction or valorization has returned ever more strongly to the focus of interest. In general, metals are used as catalysts for the electrolysis of CO₂.

TABLE 1
according to the citation Y. Hori, Electrochemical CO₂ reduction
on metal electrodes, in: C. Vayenas, et al. (Eds.), Modern Aspects
of Electrochemistry, Springer, New York, 2008, pp. 89-189.

| Electrode | CH₄ | C₂H₄ | C₂H₅OH | C₃H₇OH | CO | HCOO⁻ | H₂ | Total |
|---|---|---|---|---|---|---|---|---|
| Cu | 33.3 | 25.5 | 5.7 | 3.0 | 1.3 | 9.4 | 20.5 | 103.5 |
| Au | 0.0 | 0.0 | 0.0 | 0.0 | 87.1 | 0.7 | 10.2 | 98.0 |
| Ag | 0.0 | 0.0 | 0.0 | 0.0 | 81.5 | 0.8 | 12.4 | 94.6 |
| Zn | 0.0 | 0.0 | 0.0 | 0.0 | 79.4 | 6.1 | 9.9 | 95.4 |
| Pd | 2.9 | 0.0 | 0.0 | 0.0 | 28.3 | 2.8 | 26.2 | 60.2 |
| Ga | 0.0 | 0.0 | 0.0 | 0.0 | 23.2 | 0.0 | 79.0 | 102.0 |
| Pb | 0.0 | 0.0 | 0.0 | 0.0 | 0.0 | 97.4 | 5.0 | 102.4 |
| Hg | 0.0 | 0.0 | 0.0 | 0.0 | 0.0 | 99.5 | 0.0 | 99.5 |
| In | 0.0 | 0.0 | 0.0 | 0.0 | 2.1 | 94.9 | 3.3 | 100.3 |
| Sn | 0.0 | 0.0 | 0.0 | 0.0 | 7.1 | 88.4 | 4.6 | 100.1 |
| Cd | 1.3 | 0.0 | 0.0 | 0.0 | 13.9 | 78.4 | 9.4 | 103.0 |
| Tl | 0.0 | 0.0 | 0.0 | 0.0 | 0.0 | 95.1 | 6.2 | 101.3 |
| Ni | 1.8 | 0.1 | 0.0 | 0.0 | 0.0 | 1.4 | 88.9 | 92.4 |
| Fe | 0.0 | 0.0 | 0.0 | 0.0 | 0.0 | 0.0 | 94.8 | 94.8 |
| Pt | 0.0 | 0.0 | 0.0 | 0.0 | 0.0 | 0.1 | 95.7 | 95.8 |
| Ti | 0.0 | 0.0 | 0.0 | 0.0 | 0.0 | 0.0 | 99.7 | 99.7 |

The table above shows an overview of the Faraday efficiencies for the conversion of CO₂ into various products on different metal electrodes. The Faraday efficiency (also known as the Faradaic efficiency, Faradaic yield, Coulomb efficiency or current efficiency) describes the efficiency with which charge (electrons) is transferred in a system which enables an electrochemical reaction. The word “Faraday” in this term has two mutually associated aspects. First, the historical unit of charge is the faraday, although this has since been replaced by the coulomb. Secondly, the related Faraday constant correlates the charge with moles of matter (amount of substance) and electrons. This phenomenon was originally understood through the work of Michael Faraday and expressed in his laws of electrolysis.

Table 1 shows the typical Faradaic efficiencies on different metal cathodes. Thus, CO₂ is reduced almost exclusively to CO for example on Ag, Au, Zn, with restrictions on Pd, Ga, while on copper a large number of hydrocarbons are to be observed as reduction products. Besides pure metals, metal alloys are also of interest since they can increase the selectivity of a particular hydrocarbon. In the prior art, however, there is still little specific in connection with metal alloys.

The following reaction equations represent the reactions at the anode and at the cathode for reduction on a copper cathode. The formation of expensive, i.e. economically valuable, ethylene is of particular interest in this case. The reductions on the other metals are given in a similar way thereto.

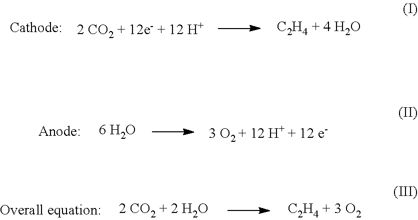

Besides this, however, there are also a range of byproducts, for example ethanol, CO, H₂, as well as formate and acetate. It has already been possible to study and demonstrate the function of an electrochemical conversion of CO₂ into usable hydrocarbons in the laboratory and pilot installations.

On the other hand, the efficient purification of the product flows from the conversion reactions, which is necessary for industrial use, has not yet been resolved.

Although these so-called downstream processes for the industrial production of ethylene from fossil raw materials (for example hydrocracking) have been known for many years and substantially optimized, these methods cannot however, or cannot readily, be applied for the purification of products from the electrochemical conversion of CO₂ because of the entirely different composition of the product flows.

### SUMMARY OF INVENTION

Against this background, the object of the invention is to provide a method with which efficient separation, and as far as possible specific selection, of desired products from CO₂ electrolysis can be achieved on the industrial scale.

A further object is to provide a corresponding apparatus which is configured to carry out efficient separation and selection of products from CO₂ electrolysis.

The object aimed at a method is achieved according to the invention by a method for processing products from a CO₂ electrolysis, wherein carbon dioxide CO₂ and water are electrochemically converted in a cathode space of an electrolysis cell, wherein gaseous cathode products which comprise at least ethylene, hydrogen and carbon monoxide are formed, wherein the gaseous cathode products are processed in a multistage separation process. The method is characterized by the following steps: in a first step, the cathodic product gas flow is delivered to desublimation so that CO₂ and water are frozen out from the product gas flow and separated. In a second step the product gas flow purified with respect to CO₂ is compressed to a pressure. In a third step, the compressed product gas flow is delivered to gas permeation, hydrogen in the product gas flow being separated by passing the hydrogen through a hydrogen-permeable membrane. In a fourth step, the retentate remaining in the product gas flow, containing ethylene and carbon monoxide, is subjected to separation by distillation, so that ethylene and carbon monoxide are separated.

The invention proposes a particularly advantageous and mutually attuned multistage separation sequence which particularly effectively utilizes the particular physical properties of the materials involved in order to achieve a maximally energy- and cost-efficient method for the purification and extraction of ethylene as a preferred final product.

By the multistage separation method, ethylene with high purity can particularly advantageously be obtained from the gaseous products of a CO₂ electrolysis with high selectivity. The sequence of the separation methods respectively applied is also selected advantageously in the method.

Preferably, the product gas flow is compressed to a pressure of from 10 bar to 50 bar, in particular to 45 bar, in the third step. An advantageous volume flow reduction is achieved in this way, all the more so since CO₂ and water have already been separated beforehand by freezing out from the product gas flow in the first step.

Further preferentially, compressed retentate from the membrane separation is fed for separation by distillation into a rectifying column, ethylene being obtained in the liquid phase and carbon monoxide being obtained in the gas phase. From the large number of components in the product gas flow, ultimately valuable ethylene with high purity is therefore obtained, which is formed as an electrochemical conversion path from the CO₂ electrolysis as a product.

In the method, ethanol is preferentially furthermore formed as a cathode product, ethanol being condensed out and extracted in the liquid phase. A further valuable material is therefore available, namely ethanol, which is selectively separated.

Preferably, formate and/or acetate in liquid or dissolved form are furthermore formed as cathode products, and are separated from the product gas flow in a separation method.

The object relating to an apparatus is achieved according to the invention by an apparatus for processing a product gas flow from a CO₂ electrolysis, containing at least ethylene, hydrogen and carbon monoxide as gaseous cathode products, having a desublimation unit for freezing CO₂ and water out from the product gas flow, having a compressor unit downstream of the desublimation unit, comprising a compressor, and having a hydrogen-permeable membrane unit downstream of the compressor unit for separating hydrogen from the product gas flow.

In a particularly preferred embodiment, the apparatus is equipped with a cryodistillation unit downstream of the hydrogen-permeable membrane unit.

Further preferentially, the compressor unit has a cooling apparatus downstream of the compressor, by means of which the compressed product gas flow can be cooled and the heat of compression can be dissipated.

Further preferentially, the cryodistillation unit has a rectifying column, at the head of which a condenser that is designed to condense out ethylene is arranged.

Preferably, trays or packings are introduced inside the rectifying column, which cause intensive contact of ascending gas and downflowing liquid in counterflow so that successive enrichment of ethylene in the liquid phase and corresponding enrichment of CO in the gas phase can be achieved.

In one particularly preferred embodiment, the rectifying column has a column bottom which is designed in such a way that liquid ethylene with high purity can be extracted at the column bottom. The ethylene can therefore be removed selectively from the installation forming the overall apparatus and be employed as a valuable material for other purposes.

In this case, a heating unit is preferentially arranged at the column bottom, by means of which a part of the liquid flow arriving in the column bottom can be evaporated so that a continuous counterflow of gas and liquid can be achieved in the rectifying column.

In a particularly preferred embodiment, the apparatus can be connected to a CO₂ electrolysis installation via a connection unit so that a product gas flow from the CO₂ electrolysis can be delivered to the apparatus. In this case, a connection unit for the cathode products may be provided on the cathode side and a connection unit for the anode products may be provided on the anode side.

The invention is already based on the discovery that, because of the particular functional principle of a CO₂ electrolysis, different electrochemical conversion reactions take place in a respective cathode space and anode space, which are spatially separated from one another, of a CO₂ electrolysis cell or in an electrolyzer comprising a large number of CO₂ electrolysis cells, and these are to be taken into account for an efficient separation and purification method. Under the normal reaction conditions, gaseous and condensed products are in this case formed, and a distinction may essentially be made between four product flows that leave a cell, or an electrolyzer:
gaseous products from the cathode space
catholyte with products dissolved therein
gaseous products from the anode space
anolyte with products dissolved therein.

Various possibilities for the more detailed functional embodiment and design structure of an electrolysis are described in the literature. Various options are therefore available for constructing and operating a CO₂ electrolysis cell. The method and the apparatus of the invention may be applied particularly flexibly to the different cell designs and modes of operation for CO₂ electrolysis.

The CO₂ electrolysis cell may therefore comprise various designs and modes of operation, combinations and variants also being possible. They are therefore not to be understood as restrictive and respectively exclusive, but in turn admit different subvariants. The different variants of the CO₂ electrolysis cells and their operation are known in principle in terms of their basic functions. A so-called flow cell architecture (two gaps), a one-gap architecture or a zero-gap architecture may be envisioned here as a structure for the CO₂ electrolysis cell, and these cell designs are advantageously applied in the scope of the invention as an upstream process of the purification.

The separation and processing method of the invention is composed of these basic processes, utilizes them and is flexibly applicable and respectively adaptable to various cell designs and modes of operation of the electrolysis cell. When applying its application, the method may therefore respectively differ specifically in respect of the processing of the electrolyte and of the gases produced on the cathode and anode sides in the CO₂ electrolysis cell.

A feature common to all alternative embodiments of CO₂ electrolysis is so-called flow-by operation i.e. the CO₂ sweeps past the gas diffusion electrode on the side facing away from the electrolyte. The product gases are thereby produced in this gas space and are removed with excess CO₂. The products are subsequently delivered to separation and processing methods of the invention.

The variants differ—as described above—primarily by the number of gaps, or reaction regions, in the electrolysis cell and in the use of different membrane types. Inter alia, the following reactions take place at the cathode:

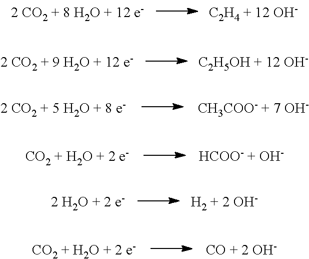

For the neutral molecules such as ethylene, ethanol or CO, a corresponding number of hydroxide ions according to the electrons required are formed on the product side. For singly negatively charged species, there is one hydroxide ion less, i.e. the charge is compensated for by the resulting anion.

The hydroxide ions react with excess CO₂ to form carbonate or hydrogen carbonate, which is released into the electrolyte, according to:

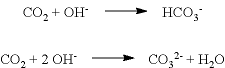

Formate and acetate are correspondingly enriched and need to be separated from the carbonate-containing electrolyte. Salt separation methods are used for this. According to the invention, a separation of the distillable products such as ethanol or ethylene is carried out in a particularly advantageous way.

In the 2-gap structure, CO₂ and O₂ are formed in the anode gap.

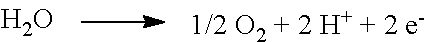

The protons released liberate the chemically absorbed CO₂ from the cathode side-reaction.

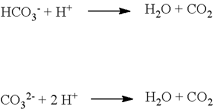

Because of the release of O₂ and CO₂ at the same place, the 2-gap cell design therefore seems of rather little interest, or uneconomical, for commercial operation since an additional separation problem is incurred.

This problem is avoided in the particularly preferred 1-gap design since the CO₂ is released in the gap and O₂ is correspondingly released on the anode side. This O₂ released on the anode side contains only a few percent of CO₂. The CO₂ released in the gap may in turn be recirculated. The 1-gap design is therefore preferred for commercial operation. Optionally (not graphically represented), an additional electrolyte circuit can follow on from the rear side of the anode. This may consist of water or acid. Salts require additional electrolyte management in order to control the cation equilibrium in the electrolyte. In the method of the invention, the electrochemical conversion of carbon dioxide and water in the cathode space is therefore preferably carried out in a 1-gap configuration.

The 0-gap architecture (“zero-gap architecture”) of a CO₂ electrolysis cell contains an anion-conducting membrane. Besides the hydroxide ion shown, hydrogen carbonate, carbonate, formate or acetate are also ionic charge carriers that move to the anode. There, they in turn release CO₂ and are neutralized to form formic acid or acetic acid. Alternatively, formate, formic acid, acetate or acetic acid may be oxidized back into CO₂, which is rather detrimental to the overall efficiency.

In a particularly preferred embodiment of the separation and processing method of the invention, a cell design in a 1-gap architecture is preferably adopted. This is advantageous primarily because of its inherent CO₂ separation in the gap. Other cell arrangements, including combined cell arrangements, for example with a combination of anion-conducting and cation-conducting or bipolar membranes, are alternatively possible depending on the application. These, however, are generally less preferred because of their complexity, or in some cases lower energy efficiency.

In practice, the separation of the liquid or dissolved products and the gaseous products from an electrolysis is preferentially carried out in a respective gas-liquid phase separator, into which the two-phase catholyte flow and the anolyte flow are conveyed. The catholyte flow is in this case introduced into a first gas-liquid phase separator and the anolyte flow is introduced into a second gas-liquid phase separator. During the electrolysis, gaseous anode products and liquid anode products are formed on the anode side. Correspondingly, gaseous cathode products and liquid cathode products are formed on the cathode side. The gaseous cathode products are of particular interest for processing and separating ethylene in particular as a high-value product in the catholyte flow by the method of the invention.

Degassed catholyte and degassed anolyte are preferably recirculated into the electrolysis cell. This circuit is simultaneously also used to dissipate the resulting process heat-via suitably designed heat exchangers. In the case of the electrochemical conversion of CO₂ into hydrocarbons, CO₂ is introduced as a starting material into the cathode space.

As may be seen from Table 1, a product spectrum is formed during the electrochemical conversion, which essentially contains methane (CH₄), ethylene (C₂H₄), hydrogen (H₂), carbon monoxide (CO), ethanol (C₂H₅—OH), propanol (C₃H₇—OH) and formate (HCOO⁻), but as is known also comprises acetate (H₃CCOO⁻).

In the gas flow of the cathode side, during electrolysis in an electrolyzer having a large number of electrolysis cells, ethylene, hydrogen and CO are preferably formed as gaseous cathode products. Furthermore, this product flow also contains a large amount of CO₂, which is run in a superstoichiometric ratio in the electrolysis. Formed in the gaseous product flow as further constituents are water vapor as well as the other liquid products according to their saturated vapor pressure under the conditions in the respective gas-liquid phase separator for the catholyte flow and the anolyte flow.

The obtaining or separation of ethylene as a product is particularly preferential in a CO₂ electrolysis operated in this way. In order to obtain a commercially usable product, it is necessary to separate ethylene from this gas flow in a form that is as pure as possible.

For this, the invention proposes an advantageous multistage and adapted separation sequence which effectively utilizes the particular physical properties of the materials involved in order to achieve a maximally energy- and cost-efficient method for the purification and extraction of ethylene as a preferred final product. The electrochemical reduction of CO₂ into hydrocarbons, in particular the valuable chemical raw material ethylene C₂H₄ (˜1000 €/t) has been described in the literature since the 1990s. In recent years, the research activities have increased greatly because the storage/use of this energy seems expedient in economic terms because of the availability of surplus electrical energy from nonfossil generation sources such as solar or wind.

The present invention describes a particularly energy-efficient method for generating products that are usable in economic and practical terms from a CO₂ electrolysis process with sufficient purity. Such a method has not previously been known. The technical feasibility of the proposed method management and the plant-specific implementation are available and have been verified and confirmed with the aid of reliable modern process simulation tools. In the scope of the process synthesis, a range of obvious conventional separation sequences have also been studied, for example classical thermal separation of the gas constituents according to the boiling sequence. These, however, have been found to be energetically unfavorable or technically/physically unrealistic because of the specific material or system properties. Because of the small difference in the volatilities, for example, separation of CO₂ and ethylene by distillation is very expensive, or probably not even possible by means of simple distillation owing to the formation of an azeotrope, which is predicted in the process simulation. Here, the method and the apparatus of the present invention are superior.

This being the case, a separation sequence is particularly advantageously proposed in which a very effective combination of special method steps is provided, particular advantages resulting here from the special component and system properties.

Further, the proposed method steps surprisingly solve the problem of the desired recirculations and necessary discharges of constituents very economically and efficiently, so that a highly resource-saving process is at the same time provided, only a small fraction of unusable waste products or emissions being generated.

The advantages of the method according to the invention and of the apparatus according to the invention may be attributed mutatis mutandis to one another.

Further advantages, features and details of the invention may be found in the following description of preferred exemplary embodiments and with the aid of the drawing. The features and feature combinations mentioned above in the description, as well as the features and feature combinations mentioned below in the description of the figures and/or shown only in the individual figures, may be used not only in the combination respectively indicated but also in other combinations or individually, without departing from the scope of the invention.

### BRIEF DESCRIPTION OF THE DRAWINGS

Exemplary embodiments of the invention will be explained in more detail below with the aid of a drawing, in which, schematically and in a highly simplified manner:

FIG. 1 shows an exemplary embodiment of an apparatus for processing the products from a CO₂ electrolysis in various separation steps;

FIG. 2 shows an exemplary embodiment of an overall installation comprising a CO₂ electrolysis installation and a processing apparatus for electrolysis products downstream on the product flow side.

### DETAILED DESCRIPTION OF INVENTION

Identical reference signs have the same meaning in the figures.

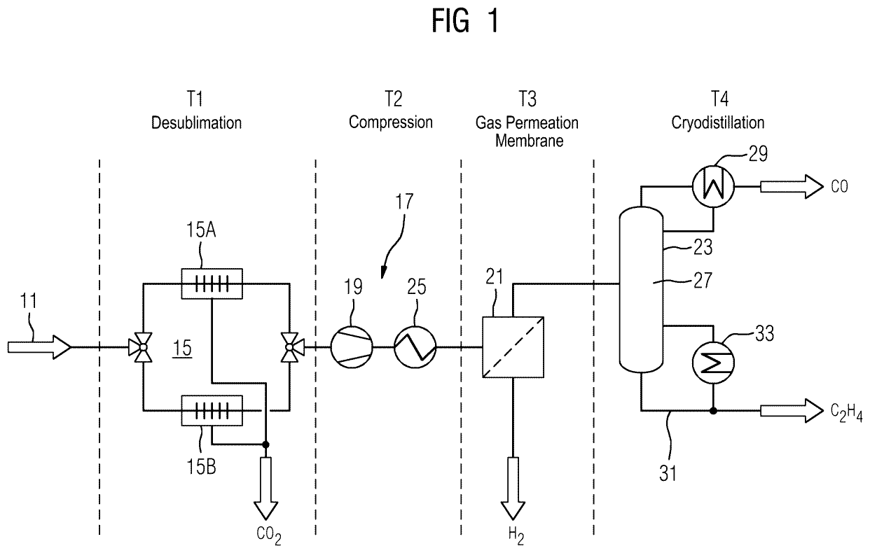

FIG. 1 represents an exemplary embodiment of this separation step sequence according to the invention. It shows an apparatus for processing the products from a CO₂ electrolysis in various separation steps. The separation, or purification, of the gaseous products on the cathode side of a CO₂ electrolysis cell or electrolysis installation is shown in more detail in a schematic representation here. The input flow 11 into this separation sequence comprises gaseous cathode products and consists to a large extent of CO₂ (about 80 m %). As secondary components, approximately 5 m % carbon monoxide CO and about 0.5-1 m % hydrogen H₂ are present. As the final product, it contains ethylene C₂H₄ in a proportion of about 3-4 m %. The input flow 11 is saturated with water H₂O at about 1 bar and 60° C., which corresponds to a content of about 10 m %.

In the method, it has been found particularly preferential to separate carbon dioxide CO₂ and water H₂O initially in a first separation step T1. In this desublimation step, selective separation is advantageously possible by cooling the gaseous input flow 11 to such an extent that water H₂O and carbon dioxide CO₂ freeze out. For this purpose, a process temperature below about −80° C. is provided in the separation step T1, this being possible even without energy- and cost-intensive compression. The freezing may in the simplest case for example take place in a cooled container in which dry ice and frozen water accumulate during the cooling.

The container or reactor of the desublimation unit 15 is preferably configured redundantly so that it is possible to switch over to a second container 15B when the holding capacity of a first container 15A is reached, which enables continuous operation. CO₂ and water that have been frozen out can then be returned into the gas state by heating, and in this way a previously filled desublimation container 15A, 15B can be emptied again. Released CO₂ and water H₂O may be reused and are preferably recirculated back into the electrolysis process.

A further advantage of this procedure is that the product mass flow to be processed in this step is greatly reduced since carbon dioxide CO₂ and water H₂O together make up about 90 m % of the input flow 11 into the desublimation unit 15 here. This is important and advantageous insofar as the remaining reduced gaseous product mass flow is compressed to 10-50 bar in the following step T2 of the processing. For this purpose, a compressor unit 17 is provided, which has a compressor 19. Because of the reduced product mass flow, the following process step T2 can be carried out with much less plant-specific outlay and energy expenditure in respect of the compressor performance of the compressor 19.

The heating that results from the compression in the compressor 19 in separation step T2 is preferably compensated for by suitable cooling, so that the gaseous product mass flow is kept at a temperature of about 60° C. For this purpose, the compressor unit 17 has a cooling apparatus 25 which is connected downstream of the compressor 19 in the flow direction of the product mass flow.

Subsequent to the compression and cooling in the compressor unit 17, the next separation step T3 takes place in the form of gas permeation. This step purposely makes use of the fact that hydrogen is a very small molecule and therefore exhibits a comparatively high permeability by suitable use of gas permeation membranes. For this purpose, a hydrogen-permeable membrane unit 21 comprising a membrane is connected fluidically downstream of the compressor unit 17. Hydrogen H₂ is therefore separated very efficiently from the product gas flow in the separation step T3, which is carried out in the gas permeation step T3 of the method. Hydrogen H₂ that passes selectively through the hydrogen-permeable membrane is preferentially likewise recirculated into the upstream CO₂ electrolysis process. As an alternative, particularly in the case of an undesired buildup, it is possible that this hydrogen gas flow or a part thereof is also discharged from the installation. Hydrogen is a gas which is inert for the electrochemical reaction per se, and actually promotes the diffusion of the production. After the buildup, it is preferentially separated and used separately. Since the membrane-based separation in step T3 does not take place fully selectively and the other volatile secondary components can also pass through the hydrogen-permeable membrane to a certain-albeit minor-extent, this material flow may be used as output so that secondary components do not become undesirably highly concentrated in the process.

The remaining retentate from the separation step T3 now contains predominantly carbon monoxide CO and ethylene C₂H₄, of which the difference in volatility is large enough to carry out separation by distillation in a step T4 by means of a cryodistillation unit 23 at a suitably low temperature. For this purpose, the already compressed retentate flow from the membrane separation is introduced into a rectifying column 27, which in a preferred embodiment of the method is adjusted and operated cryogenically, i.e. at about 50 bar and with about −100° C. at the column head, or is operated with about +6° C. in the column bottom.

A condenser 29 is fitted at the head of the rectifying column 27 and cools the ascending gas to such an extent that high-boiling ethylene C₂H₄ is condensed and recirculated into the rectifying column 27. The condenser 29 works only partially, and the resulting carbon monoxide gas flow in the condenser 29 is recirculated as gas into the electrolysis, or it is possible that here again a partial flow is delivered to thermal valorization. Carbon monoxide is advantageously reduced on the same catalyst likewise to form ethylene C₂H₄ or ethanol C₂H₅OH.

Inside the rectifying column 27, there are fixtures—for example so-called trays or packings—which cause intensive contact of ascending gas and downflowing liquid according to a counterflow principle. This process leads to successive enrichment of ethylene C₂H₄ in the liquid phase and corresponding enrichment of carbon monoxide CO in the gas phase.

In this way, ethylene C₂H₄ can be obtained with high purity at the column bottom 31 of the rectifying column 27. In order to sustain the rectifying process T4 in the region below the gaseous feed entry as well, a part of the liquid flow arriving in the column bottom 31 may be evaporated with the aid of a heater unit 33 in order to ensure and continuously maintain a continuous counterflow of gas and liquid during operation.

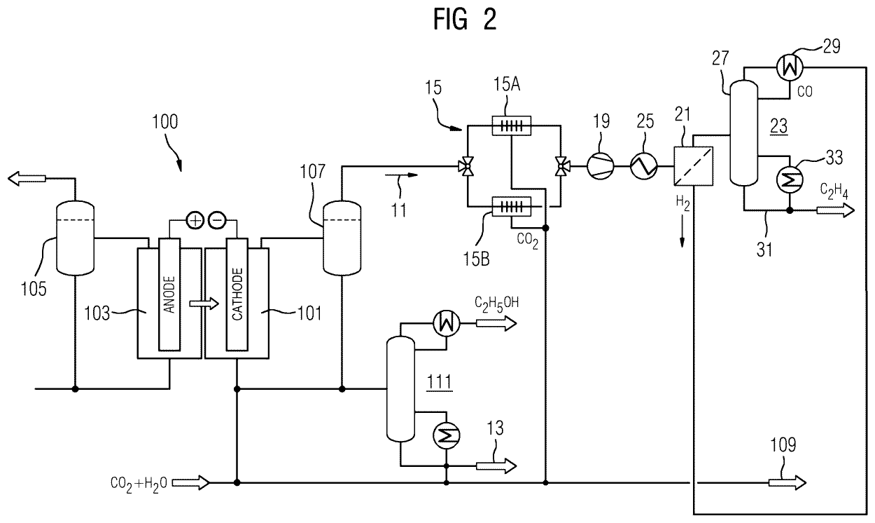

As a further valuable product during the electrolysis of carbon dioxide CO₂, ethanol C₂H₅OH is formed, which under the operating conditions of the electrolysis enters the multicomponent material flow after the gas separator 107 predominantly as a liquid. When connecting an electrolysis installation to the downstream apparatus for processing the product gas flow, it is possible and advantageous to introduce a partial flow from this material flow into a further distillation column 111. Such an interconnection is shown by way of example in FIG. 2. Here, ethanol C₂H₅OH may be obtained in an azeotropic composition as a head product in the distillation column 111. The bottom product is recirculated into the CO₂ electrolysis.

A partial flow 13 may in this case be discharged in order to purposely remove further byproducts (for example formate and acetate), which occur in liquid or dissolved form, from the overall process.

FIG. 2 shows a schematic overview which is based on the method steps described above, so that a preferred integrated overall method is achieved, which comprises the CO₂ electrolysis on the one hand and on the other hand the subsequent purification of the products in the substeps of desublimation T1, compression T2, membrane-based separation T3 and cryodistillation T4.

FIG. 2 in this case shows a schematic representation of the overall method in a flowchart of the process of an electrochemical production of ethylene C₂H₄ and ethanol C₂H₅OH from CO₂ and water H₂O and the separation of the cathode-side gaseous products according to the invention.

Here, as already described, in an electrolysis installation 100 or electrolysis cell, water H₂O and carbon dioxide CO₂ are initially converted electrochemically in the cathode space 101 essentially into ethylene, hydrogen, carbon monoxide, ethanol, formate and acetate. Oxygen is essentially formed in the anode space 103, and is separated from the anolyte with a sufficient purity with the aid of a gas-liquid separator 105.

The catholyte is fed with the reaction products likewise into a suitable gas-liquid separator 107, and the resulting gas and liquid flows are processed separately. This cathodic product gas flow 11 contains about 80 m % carbon dioxide CO₂. As secondary components, approximately 5 m % carbon monoxide CO and about 0.5-1 m % hydrogen are present. As the final product for the processing and separation, it contains ethylene C₂H₄ in a proportion of about 3-4 m %. The cathodic product gas flow 11 is saturated with water at about 1 bar and 60° C., which corresponds to a content of about 10 m %.

This product gas flow 11 is also referred to as a feed flow, and is initially delivered into the desublimation unit 15 where the gas is cooled to at least −80° C., so that CO₂ and water H₂O are frozen out from the product gas flow 11. The desublimation is configured redundantly by means of two containers, a first container 15A and a second container 15B. It is therefore possible to switch over when the holding capacity of one of the containers 15A, 15B is reached, and continuous operation is therefore enabled. CO₂ and water H₂O that have been frozen out can then be returned into the gas state by heating, and the previously filled container 15A or 15B may thus be emptied. Released CO₂ and water H₂O may be reused and are recirculated into the electrolysis process of the electrolysis installation 100.

The product gas flow 11 dried and freed from CO₂ in step T1 (cf. FIG. 1) is compressed in step T2 in the compressor 19 to 45 bar and regulated to 60° C., and then introduced into the membrane unit 21 in order to carry out a gas permeation, where hydrogen preferentially passes through the membrane and is thus separated selectively from the product gas flow 11. This hydrogen flow is preferentially likewise recirculated into the electrolysis installation 100, or in the event of an undesired high concentration, this hydrogen flow or a part thereof may also be discharged from the process. Since the membrane separation in the membrane unit 21 generally does not take place fully selectively and the other volatile secondary components 109 can therefore also pass through the membrane to a minor extent, this material flow 109 may be used as output so that secondary components do not build up in the process to an undesired extent.

The remaining product flow-predominantly comprising or consisting of carbon monoxide CO and ethylene C₂H₄—is separated with the aid of a cryorectifying column 27 having a separating performance of about 10 theoretical plates in the rectifying part and about 20 theoretical plates in the stripping part. For sufficient separation, a reflux ratio of two is required. With a column pressure of 45 bar, condensation takes place at the column head with a temperature of −100° C. and the column bottom is regulated to +5.6° C. This results in ethylene at the column bottom 31 with a very high purity of 99.98 vol %.

The condenser 29 works only partially, and the resulting carbon monoxide flow in the condenser 29 is recirculated as gas into the electrolysis installation 100 or may also be delivered as a partial flow to another type of valorization, particularly in the case of an excessive concentration in the process.

The liquid catholyte flow which leaves the cathode-side gas separator 107 contains as a further valuable product about 10 m % ethanol C₂H₅OH. Besides further byproducts (acetate and formate <1%), the catholyte essentially contains 73 m % water, 11 m % potassium hydrogen carbonate and 7 m % potassium sulfate. This material flow is introduced into a further rectifying column 111, comprising a rectifying part with about 15 theoretical plates and a stripping part with about 30 theoretical plates, where an ethanol/water mixture having an azeotropic composition is obtained by distillation as the head product. This further rectifying column 111 is operated at atmospheric pressure with a reflux ratio of two. The condensation temperature is 78° C., and the reboiler is heated to 100° C. The catholyte freed from ethanol C₂H₅OH is obtained as the bottom product and recirculated into the electrolysis installation.

In order to remove further byproducts (for example formate and acetate), which occur in liquid or dissolved form, from the overall process, a partial flow 13 of the bottom product is discharged and delivered to further valorization.

1. A method for processing products from a CO₂ electrolysis, wherein carbon dioxide CO₂ and water are electrochemically converted in a cathode space of an electrolysis cell, wherein gaseous cathode products which comprise at least ethylene (C₂H₄), hydrogen (H₂) and carbon monoxide (CO) are formed, wherein the gaseous cathode products are processed in a multistage separation process, comprising:
(T1) in a first step, delivering a cathodic product gas flow to desublimation so that CO₂ and water are frozen out from the product gas flow and separated,
(T2) in a second step, compressing the product gas flow purified with respect to CO₂ to a pressure,
(T3) in a third step, delivering the compressed product gas flow to gas permeation, hydrogen (H₂) in the product gas flow being separated by passing the hydrogen (H₂) through a hydrogen-permeable membrane, and
(T4) in a fourth step, subjecting the retentate remaining in the product gas flow, containing ethylene (C₂H₄) and carbon monoxide (CO), to separation by distillation, so that ethylene (C₂H₄) and carbon monoxide (CO) are separated.

2. The method as claimed in claim 1,
wherein the product gas flow is adjusted to a pressure of from 10 bar to 50 bar, in the third step (T3).

3. The method as claimed in claim 1,
wherein compressed retentate from the membrane separation in a membrane unit is introduced for separation by distillation (T3) into a rectifying column, ethylene (C₂H₄) being obtained in the liquid phase and carbon monoxide (CO) being obtained in the gas phase.

4. The method as claimed in claim 1,
wherein ethanol (C₂H₅OH) is formed as a cathode product, ethanol (C₂H₅OH) being condensed out and extracted in the liquid phase.

5. The method as claimed in claim 1,
wherein formate and/or acetate in liquid or dissolved form as byproducts are furthermore formed as cathode products, these byproducts being separated.

6. An apparatus for processing a product gas flow from a CO₂ electrolysis, containing at least ethylene (C₂H₄), hydrogen (H₂) and carbon monoxide (CO) as gaseous cathode products, comprising:
a desublimation unit for freezing CO₂ and water out from the product gas flow,
a compressor unit downstream of the desublimation unit, comprising a compressor, and
a hydrogen-permeable membrane unit downstream of the compressor unit for separating hydrogen from the product gas flow.

7. The apparatus as claimed in claim 6,
wherein the desublimation unit is configured redundantly, a first desublimation container and a second desublimation container connected in parallel therewith being provided.

8. The apparatus as claimed in claim 6, comprising:
a cryodistillation unit downstream of the hydrogen-permeable membrane unit.

9. The apparatus as claimed in claim 6,
wherein the compressor unit has a cooling apparatus downstream of the compressor, by means of which the compressed product gas flow be is cooled and heat of compression is dissipated.

10. The apparatus as claimed in claim 8,
wherein the cryodistillation unit has a rectifying column, at the head of which a condenser that is designed to condense out ethylene (C₂H₄) is arranged.

11. The apparatus as claimed in claim 10, comprising:
trays or packings inside the rectifying column, which cause intensive contact of ascending gas and downflowing liquid so that successive enrichment of ethylene (C₂H₄) in the liquid phase and corresponding enrichment of carbon monoxide (CO) in the gas phase is achieved.

12. The apparatus as claimed in claim 10,
wherein the rectifying column has a column bottom which is designed in such a way that liquid ethylene (C₂H₄) with high purity is extracted at the column bottom.

13. The apparatus as claimed in claim 12, further comprising:
a heating unit arranged at the column bottom, by means of which a part of the liquid flow arriving in the column bottom is evaporated so that a continuous counterflow of gas and liquid is achieved in the rectifying column.

14. The apparatus as claimed in claim 6, further comprising:
a connection unit for receiving a product gas flow from a CO₂ electrolysis.

15. An arrangement comprising:
an apparatus as claimed in claim 14, and
a CO₂ electrolysis installation connected to the apparatus via the connection unit, so that a product gas flow from the electrolysis, containing gaseous cathode products, is delivered to the apparatus for purification.

16. The method as claimed in claim 2,
wherein the product gas flow is adjusted to a pressure of from 10 bar to 45 bar, in the third step (T3).
